(19) United States
(12) Patent Application Publication
(10) Pub. No.: US 2014/0107589 A1
(43) Pub. Date: Apr. 17, 2014

(54) ELECTROMAGNETICALLY ACTUATED VALVE AND RELATED METHODS OF USE

(71) Applicants: Farid Amirouche, Highland Park, IL (US); Matthew L. Cantwell, North Brook, IL (US)

(72) Inventors: Farid Amirouche, Highland Park, IL (US); Matthew L. Cantwell, North Brook, IL (US)

(21) Appl. No.: 13/654,226

(22) Filed: Oct. 17, 2012

(51) Int. Cl. A61M 5/168; F16K 31/06

(52) U.S. Cl. 604/256; 251/129.01; 251/337

(57) ABSTRACT

A valve assembly may include a valve body defining a housing for receiving a plunger, wherein the valve body includes an inlet channel and an outlet channel in selective fluid communication with the inlet channel. The valve assembly may also include a deflectable membrane operably coupled to the plunger, wherein the membrane is configured to selectively prevent fluid flow from the inlet channel to the outlet channel. Furthermore, the valve assembly may include an electromagnet for controlling a position of the plunger.

### FIELD OF THE INVENTION

The present disclosure relates to the general control of the flow of fluids, such as, e.g., liquid medicaments, to and from a pump. In particular, the present disclosure relates to metering one or more medicaments stored in one or more reservoirs during delivery to a patient.

### BACKGROUND OF THE INVENTION

Conventional valves, such as, e.g., microvalves, generally do not provide the necessary flow control required for the precise delivery of medicament to a patient. In one conventional approach, a device utilizes a spring that could be actuated with an electrical charge. This charge causes the spring to deflect a membrane into a sealing edge so as to obstruct the passage of fluid. Alternatively, the spring may deflect in the opposing direction to allow fluid flow through the valve. The main disadvantage to this approach is the amount of space available for the fluid cavity when the membrane is actuated is sometimes not large enough for sufficient flow volumes.

Other conventional valves rely on a spring to provide a normally closed or normally open operation. An additional solenoid is required to provide actuation allowing the valve to transition to the other energized state. The main disadvantage with this design is the reliance on continuous energy for maintaining the actuated state which greatly reduces the efficiency of the valve and limits the scope of battery powered portable applications. These valves also occupy a large footprint compared to the overall size of the devices they are used in.

Other technologies which specifically focus on reducing the size of the valve footprint rely on expensive microfabrication techniques which are difficult to implement and have relatively low yields, thereby making them too costly for mass production. Furthermore, the materials used in these devices are costly and require extraordinarily high tolerances for correct operation. Thus, there exists a need for a cost-effective and energy-efficient valve capable of accurately metering the flow of medicaments from one or more reservoirs,

### SUMMARY OF THE INVENTION

Embodiments of the present disclosure relate to, among other things, the general control of medicament delivery from a reservoir to a patient. Each of the embodiments disclosed herein may include one or more of the features described in connection with any of the other disclosed embodiments.

In one embodiment, a valve assembly may include a valve body defining a housing for receiving a plunger, wherein the valve body includes an inlet channel and an outlet channel in selective fluid communication with the inlet channel. The valve assembly may also include a deflectable membrane operably coupled to the plunger, wherein the membrane is configured to selectively prevent fluid flow from the inlet channel to the outlet channel. Furthermore, the valve assembly may include an electromagnet for controlling a position of the plunger.

Embodiments of the valve assembly may include one or more of the following features: the valve body may include a first housing member and a second housing member; the inlet and outlet channels may be defined in the second housing member: the inlet channel may include a plurality of inlet channels, and the outlet channel may include a plurality of outlet channels; the deflectable membrane may be disposed between the first and second housing members; a resilient member configured to bias the plunger towards the deflectable membrane; the resilient member may be disposed between the first housing member and the electromagnet; the plunger may include a plate operably coupled to a curved actuating portion; a return spring configured to bias the deflectable membrane towards the plunger; the return spring may be disposed in the second housing member; and a permanent magnet, wherein the membrane may be disposed between the permanent magnet and the plunger.

In another embodiment, a valve assembly may include a valve body defining an inlet port and a plurality of outlet ports. The valve body may further include a plurality of electromagnets, wherein each of the plurality of outlet ports may extend through one of the plurality of electromagnets. The valve body may include a permanent magnet movably disposed within the valve body, wherein the permanent magnet may be configured to divert fluid flow from the inlet port to one of the plurality of outlet ports.

Embodiments of the valve assembly may include one or more of the following features: the permanent magnet may be configured to be attracted to one of the plurality of outlet ports; upon application of an electrical pulse, the valve assembly may be configured to reverse a polarity, causing the permanent magnet to become attracted to the another of the plurality of outlet ports; and the permanent magnet may be configured to selectively prevent fluid flow through one of the plurality of outlet ports.

In another embodiment, a valve assembly may include a valve body having a first end and a second end, wherein the first end defines an inlet port and the second end defines an outlet port. The valve body may further include a magnetic plunger assembly disposed in a cavity defined by the valve body and configured to move between a first position and a second position, wherein, in the first position, the magnetic plunger assembly may be configured to prevent fluid flow into the valve body from the inlet port. The valve assembly may also include an electromagnet disposed about the cavity, wherein selective actuation of the electromagnet controls a position of the magnetic plunger.

Various embodiments of the valve assembly may include one or more of the following features: in the second position, the magnetic plunger may be configured to allow fluid to flow from the inlet port to the outlet port; in the first configuration, the magnetic plunger may be attracted to the first end of the valve body; energizing the electromagnet may cause the magnetic plunger assembly to become attracted to the second end of the valve body; the electromagnet may include a plurality of electromagnets; the electromagnet may include a plurality of electromagnets; energizing the electromagnet may reverse a polarity of the magnetic plunger; the magnetic plunger assembly may include a permanent magnet having a first end and a second end, a seal disposed at the first end, and a crown disposed at the second; the seal and the crown may be adhesively secured to the permanent magnet; the seal may include a substantially conical shape; the seal may be fabricated from a deformable material; the crown may include at least one channel configured for fluid flow therethrough; the permanent magnet, the seal, and the crown may be configured to move together within the valve body; a portion of the valve body may be fabricated from a material configured to sustain a magnetic field; a portion of the valve body may be fabricated from a high magnetic permeability material.

In another embodiment, a medicament delivery apparatus may include a first reservoir including a first medicament, a second reservoir including a second medicament different from the first medicament, a first valve configured to meter delivery of medicament from the first reservoir, and a second valve configured to meter delivery of medicament from the second reservoir. One of the first or second valves may include a valve body defining a housing for receiving a plunger, wherein the valve body includes an inlet channel and an outlet channel in selective fluid communication with the inlet channel; a deflectable membrane operably coupled to the plunger, wherein the membrane may be configured to selectively prevent fluid flow from the inlet channel to the outlet channel, and an electromagnet for controlling a position of the plunger.

In another embodiment, a medicament delivery apparatus may include a first reservoir including a first medicament, a second reservoir including a second medicament different from the first medicament, a first valve configured to meter delivery of medicament from the first reservoir, and a second valve configured to meter delivery of medicament from the second reservoir. In some embodiments, one of the first and second valves may include a valve body having a first end and a second end, wherein the first end defines an inlet port and the second end defines an outlet port; a magnetic plunger assembly disposed in a cavity defined by the valve body and configured to move between a first position and a second position, wherein, in the first position, the magnetic plunger assembly may be configured to prevent fluid flow into the valve body from the inlet port; and an electromagnet disposed about the cavity, wherein selective actuation of the electromagnet controls a position of the magnetic plunger.

In one embodiment, when the magnetic plunger assembly is in the second position, fluid may be permitted to flow from the inlet port, through the valve body, and out of the outlet port.

It may be understood that both the foregoing general description and the following detailed description are exemplary and explanatory only and are not restrictive of the invention, as claimed.

### BRIEF DESCRIPTION OF THE DRAWINGS

The accompanying drawings, which are incorporated in and constitute a part of this specification, illustrate exemplary embodiments of the present disclosure and together with the description, serve to explain the principles of the disclosure.

FIGS. 1A-1B illustrate a perspective view, and cross-sectional view, respectively, of a non-contact actuated flat bottom plunger valve, in accordance with an embodiment of the present disclosure;

FIGS. 2A-2B illustrate a perspective view, and cross-sectional view, respectively, of a non-contact actuated spherical cap seal valve, in accordance with an embodiment of the present disclosure;

FIGS. 3A-3B illustrate a perspective view, and cross-sectional view, respectively, of a non-contact actuated flat bottom plunger valve with a return spring, in accordance with an embodiment of the present disclosure;

FIG. 3C illustrates a perspective view of the lower housing member including the return spring therein, in accordance with the embodiment of FIGS. 3A-3B;

FIGS. 4A-4B illustrate a perspective view, and cross-sectional view, respectively, of a non-contact actuated flat bottom plunger valve with magnetic return, in accordance with a further embodiment of the present disclosure;

FIG. 4C illustrates a magnified view of the channels incorporated within the lower housing member of the non-contact actuated flat bottom plunger valve with magnetic return, according to the embodiment of Figs, 4A-4B;

FIGS. 5A-5B illustrate a perspective view, and cross-sectional view, respectively, of a bi-stable actuated latching three-way valve, in accordance with an embodiment, of the present disclosure;

FIG. 5C illustrates an operational prototype of a micropump system having two bi-stable actuated latching three-way valves of FIGS. 5A-5B;

FIGS. 6A-6B illustrate a perspective view, and cross-sectional view, respectively, of a bi-stable actuated latching valve, in accordance with a further embodiment of the present disclosure; and

FIGS. 7A-7B illustrate perspective and cross-sectional views, respectively, of a bi-stable actuated latching proportioning valve, in accordance with another embodiment of the present disclosure.

### DETAILED DESCRIPTION OF THE PREFERRED EMBODIMENTS

### Overview

The present disclosure is drawn to, among other things, a new class of microvalves and microdiverters. The microvalves and microdiverters provide for controlling a microchannel, which allows for enhanced delivery methods. These microvalves and diverters are optimized in such a way as to not restrict the flow and work in combination with the microchannels and delivery unit to ensure that a desired flow rate is preserved. The miniature high precision microvalves and microdiverters disclosed herein can be mass produced to specific manufacturing guidelines and at costs that are relatively lower than conventional valves.

in one embodiment, a free floating permanent magnet plunger is enclosed in such a way as to ensure a seal against one of two sealing membranes located at the distal ends of a valve body. Through the use of a magnetically energized flow channel induced operation, a low resistance path can be formed at relatively small dimensions. Embodiments of the present disclosure may be fabricated with high-permeability magnetic permeability alloys (e.g., stainless steel), which facilitate miniaturization by allowing both fluid contact as well as magnetic latching capabilities. In particular, the contemplated alloys used to fabricate embodiments disclosed herein should exhibit a relative high degree of ability for sustaining magnetic fields. Some of the advantages of the alloys described herein facilitate certain functionalities of the valves described herein. The advantages include a high magnetic permeability while maintaining a low coercive force. In particular, the high magnetic permeability affords the material used the ability to provide a strong force of attraction in the presence of a magnetic field while being of a smaller physical size than a material with a low permeability. These advantages are maintained while still offering a magnetic saturation of approximately 1.6 Tesla. In addition, the described stainless steel alloy includes a chemical composition that promotes machinability, despite high levels of chrome.

The valves disclosed herein are well suited for microfluidic delivery systems. The high efficiencies offered by these valves make them especially suited for battery powered applications. The bi-stable valves disclosed herein also typically require only minimal amounts of power for state changes. The required power for such valves is generally in the order of microwatts. Furthermore, these microvalves can sustain backpressure operation within the range of 0 to 5 psi.

### Exemplary Embodiments

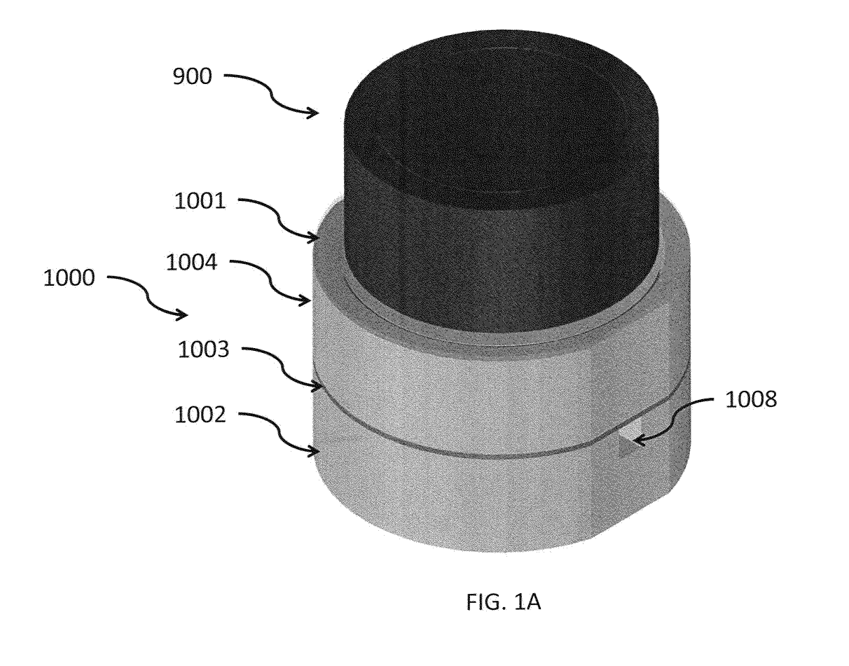

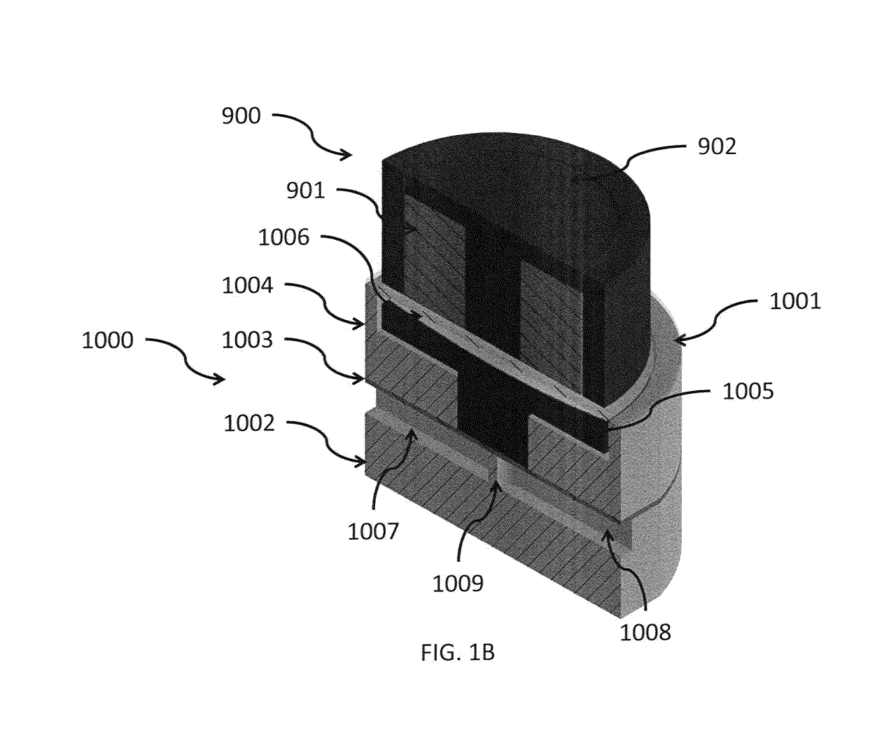

FIGS. 1A-4C illustrate embodiments of non-contact actuated valves. The depicted embodiments include an electromagnet 900 and a valve body 1000, 1100, 1200, 1300. The electromagnet 900 can be any suitable electromagnet known in the art. In some embodiments, the electromagnet 900 may be replaced with a suitable actuator, such as, e.g., a solenoid. In addition, although only one electromagnet 900 is described, any suitable number of electromagnets may be used with the embodiments discussed herein. With specific reference to FIGS. 1A-1B, the electromagnet 900 provides the necessary actuation force required to drive the moving elements of the valve body magnetically, thereby alleviating the need for direct physical contact found in conventional valve designs. The use of a magnetic driving force provides many benefits. For example, magnetic valves may allow for the more costly electromagnet to be housed in a reusable electronic assembly, while the remainder of the valve body may be disposable. The electromagnets described herein, including electromagnet 900, may be energized by any suitable electrical source, including, but not limited to, a battery or a capacitor.

With continuing reference to FIGS. 1A-1B, the electromagnet 900 includes a coil of copper magnetic wire 901, which is disposed within a high magnetic permeability housing 902. In normal operation, the housing 902 is configured to concentrate the magnetic field generated by the copper magnetic wire 901 in the direction of the valve body, thereby increasing the efficiency of the system.

The valve body 1000 depicted in FIG. 1B, e.g., includes a membrane 1003 disposed between a lower housing 1002 and an upper housing 1004. The membrane 1003 may be any suitable membrane known in the art. For example, the membrane 1003 may be fabricated of an elastomer so as to relatively flexible. In one embodiment, the membrane 1003 may exhibit elastic properties, allowing it to stretch without permanent deformation. In some embodiments, the membrane 1003 may be impermeable to fluids. In other embodiments, membrane 1003 may be impermeable to certain fluids, e.g., liquids, while being permeable to other fluids, e.g., gases.

Lower housing 1002 and upper housing 1004 may include any suitable configurations and be made of any suitable materials. For example, housings 1002 and 1004 may be fabricated from a biocompatible polymer. In some embodiments, membrane 1003 may be secured to both of housings 1002 and 1004. In other embodiments, membrane 1003 may be secured to only one of housings 1002 and 1004.

The lower housing 1002 may include a plurality of channels 1007 and 1008 which serve as a conduits for transporting fluids. The upper housing 1004 may include a plunger 1005 disposed therein. Plunger 1005 may be configured to move perpendicularly to membrane 1003 when actuated by the electromagnet 900. As depicted in FIG. 1B, the upper housing 1004 may define a channel for receiving and guiding plunger 1005.

Under normal non-energized conditions, the plunger 1005 is forced against the elastomer membrane 1003 by a spring 1006 acting from above. In some embodiments, spring 1006 may be a wave spring. In other words, spring 1006 may be configured to exert a force on plunger 1005, so that plunger 1005 is pushed into membrane 1003. The force is exerted through the membrane 1003 and down upon a sealing surface 1009 defined within lower housing 1002. The result is a sealed condition which exists between the inlet and outlet channels 1007, 1008, respectively, and thus the valve is maintained is a closed and sealed state. In other words, because spring 1006 causes membrane 1003 to be biased against sealing surface 1009, the membrane 1003 effectively creates a seal between inlet channel 1007 and outlet channel 1008, thereby preventing flow therebetween. The “inlet” and “outlet” designations are described in connection with channels 1007, 1008 respectively purely for discussion purposes. Those of ordinary skill will understand that channel 1008 may serve as an “inlet” channel, and, similarly, channel 1007 may serve as the “outlet,” according to the principles of the present disclosure.

When the electromagnet 900 is energized by passing a current through the copper magnetic wire 901, a magnetic field is induced. This magnetic field is oriented in such a way that an attractive force is developed between the electromagnet 900 and the plunger 1005 in the valve body 1000. The force is engineered to be sufficiently strong to overcome the competing forces of the spring 1006 and any ancillary frictional forces. As a result, the energized electromagnet 900 acts on the plunger 1005 causing it to move against spring 1006 until the spring 1006 is fully compressed and in a flat state. With the plunger 1005 no longer acting under the force of spring 1006, there is no longer any force acting to seal the membrane 1003 against the sealing surface 1009. Positively pressurized fluid at the inlet channel 1007 then pushes against membrane 1003 causing it to deform upwards thereby providing a path for fluid to flow from inlet channel 1007 to the outlet channel 1008.

For embodiments where the fluid in inlet channel 1007 is at a pressure greater than the pressure of outlet channel 1008 and greater than atmospheric pressure, the previously described embodiment performs very well with very fast response times, high flow rates, and relatively low forward pressure drop. However, for negative pressure operation, for which the outlet channel 1008 is at a pressure less than the pressure of inlet channel 1007 and less than atmospheric pressure, the membrane 1003 tends to be pulled closed (i.e., against sealing surface 1009) by the vacuum, thereby reducing or eliminating flow from inlet channel 1007 to outlet channel 1008. Several alternative embodiments have been devised to address the issue of efficient negative pressure operation. In the alternate embodiments described herein, the valves utilize the same electromagnet 900 described above.

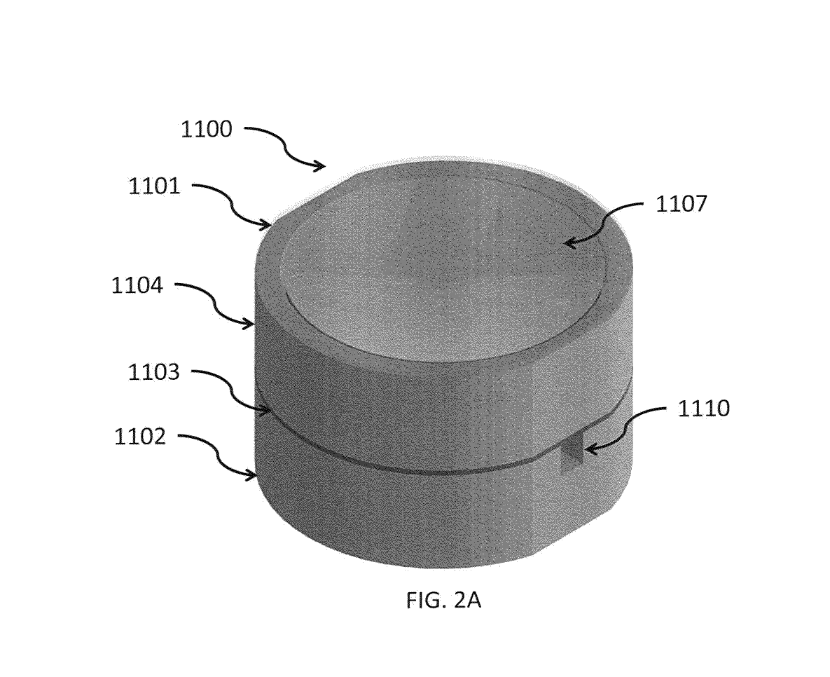

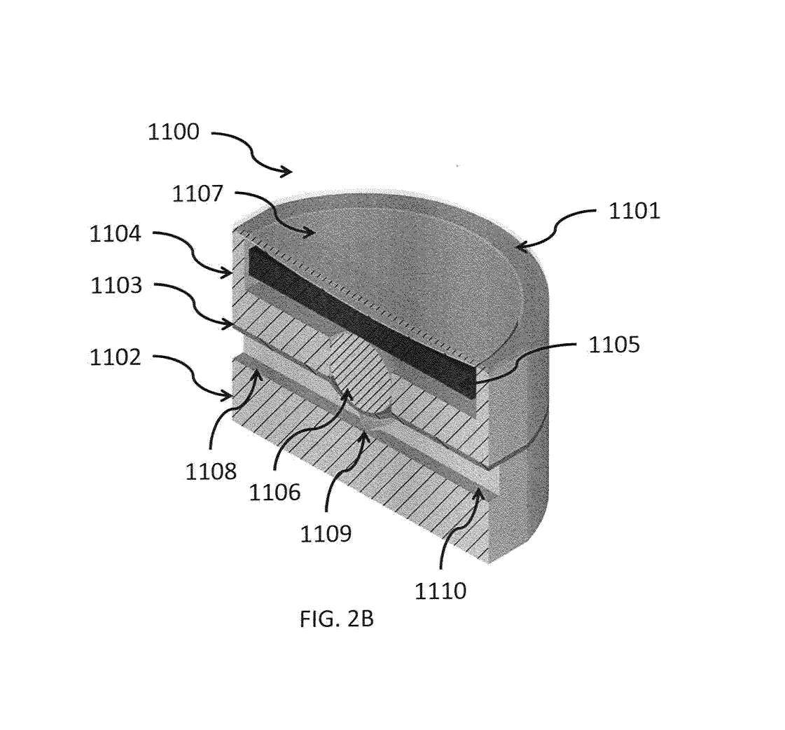

In the embodiment depicted in FIGS. 2A-2B, the plunger 1006 may be replaced with a plate 1105 having a high magnetic permeability. The plate 1105 may include a substantially planar configuration. In addition, plate 1105 may be made of any suitable material, including, but not limited to, metals, plastics, and/or alloys. The portion of the plunger 1005 which comes in contact with membrane 1003 in FIGS. 1A-1B, may be replaced with an actuating portion 1106. Although the exemplary embodiment discussed herein describes actuating portion 1106 as a sphere, those of ordinary skill in the art will understand that actuating portion 1106 may include any suitable configuration.

In addition, a semicircular channel 1109 may be formed in the lower housing member 1102. The channel 1109 may include a radius equal to the radius of the actuating portion 1106 plus the thickness of the membrane 1103. Under normal, non-energized conditions, the plate 1105 is forced against the actuating portion 1106, which is forced against the membrane 1103 by the wave spring 1107 acting from above. This force causes the membrane 1003 to seal into and against channel 1109, thereby sealing the valve and preventing fluid flow between the inlet channel 1108 and outlet channel 1110. Upon actuation of electromagnet 900 (not shown in FIGS. 2A-2B), the plate 1105 is drawn against the forces of wave spring 1107, allowing the elasticity of the membrane 1103 to force the actuating portion 1106 away from channel 1109, which provides a flow passage under both positive and negative pressure operations.

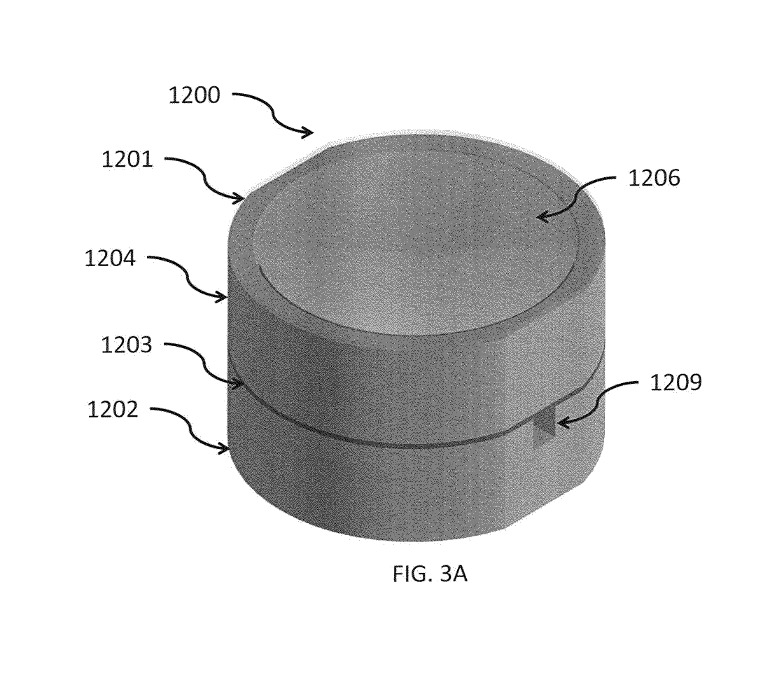

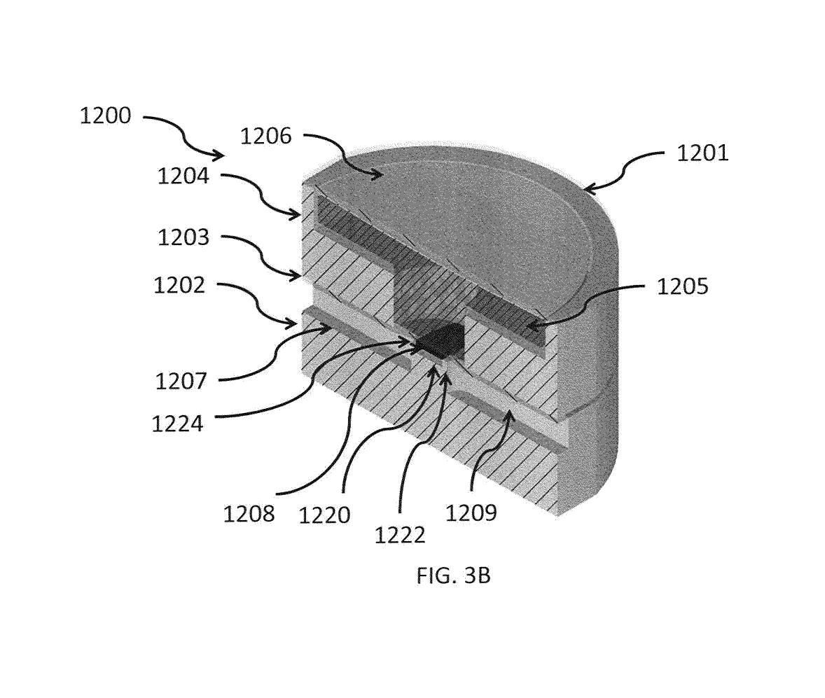

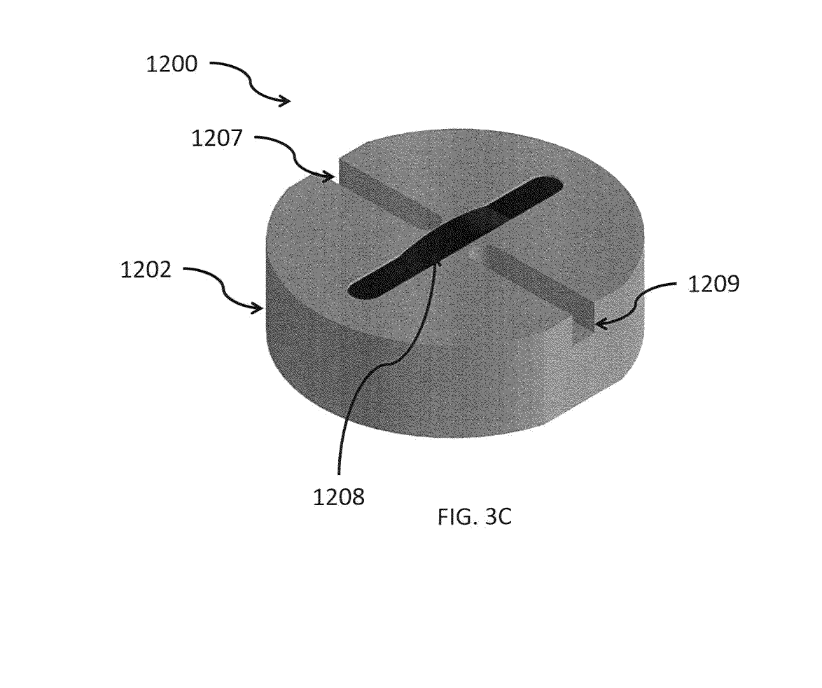

Turning now to FIGS. 3A-30, the lower housing 1202 defines a channel 1220 for housing a return spring 1208 therein. The spring 1208 may be disposed between membrane 1203 and channel 1220, as described in greater detail below. In some embodiments, the return spring may be fabricated from a suitable polymeric material. The channel 1220 includes two walls 1222 and 1224 that define respective sealing surfaces. Under normal, non-energized conditions, the plunger 1205 is forced against the membrane 1203 by a wave spring 1206 acting from above. This force causes the membrane 1203 to seal against the sealing surfaces defined by walls 1222 and 1224, thereby sealing the valve and preventing fluid flow between the inlet channel 1207 and outlet channel 1209. Upon actuation of electromagnet 900 (not shown), the plunger 1205 is drawn against the forces of spring 1206, allowing the return spring 1208 to force the membrane 1203 up in a tent-like manner and away from the sealing surfaces of walls 1222, 1224. This deformation forms a passage for fluid to flow from inlet channel 1207 to outlet channel 1209 under both positive and negative pressures. In other words, in negative pressure circumstances, the spring 1208 provides additional force (by, e.g., pushing) for moving membrane 1203 out of sealing engagement with the sealing surfaces of walls 1222 and 1224.

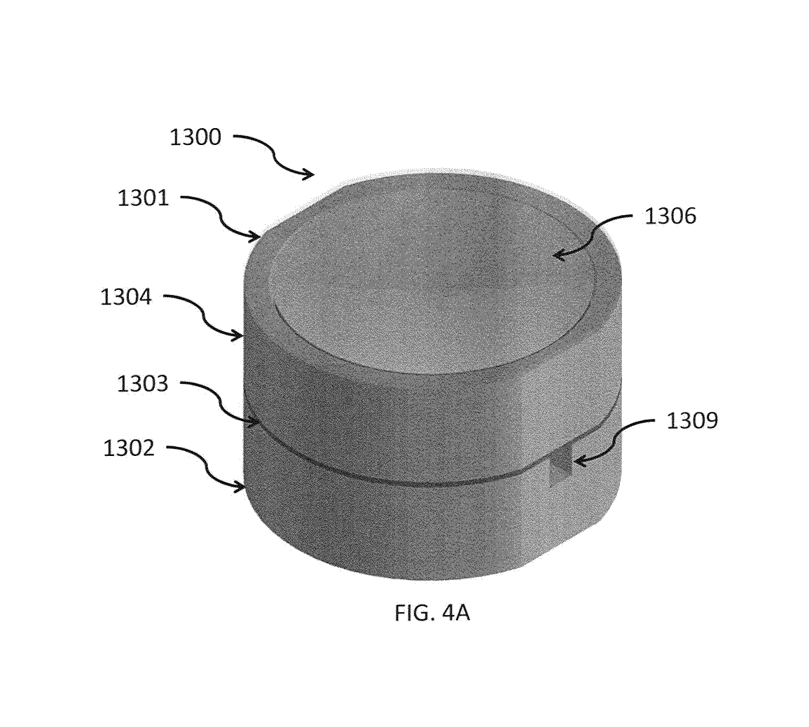

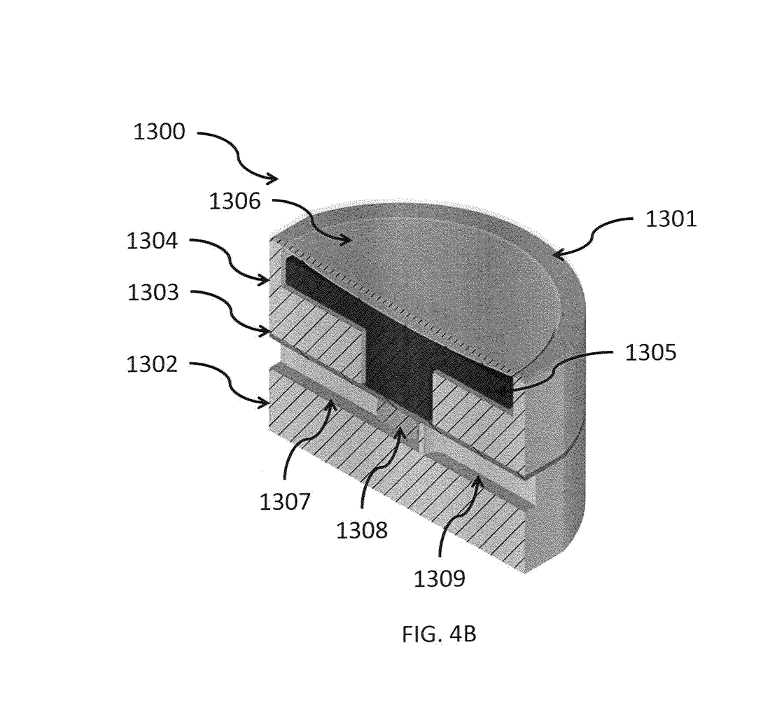

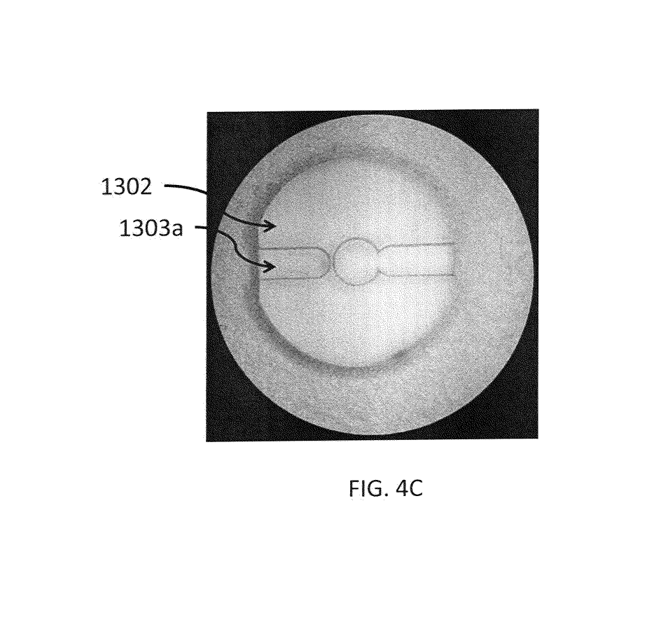

With reference now to FIGS. 4A-4C, the lower housing member 1302 may include a permanent magnet 1308 disposed therein. The permanent magnet 1308 may be configured to attract plunger 1305 towards it, and membrane 1303 may be disposed between the magnet 1308 and plunger 1305. Under normal, non-energized conditions, the plunger 1305 may be pushed against the membrane 1303 by wave spring 1306 acting from above. This causes the membrane 1303 to seal against an upper sealing surface 1303a of lower housing member 1302, thereby sealing the valve and preventing fluid flow between the inlet channel 1307 and outlet channel 1309. Upon actuation of electromagnet 900 (not shown), for example, plunger 1305 may be drawn against the force exerted by wave spring 1306 causing wave spring 1306 to become compressed. At the same time, the motion of the plunger 1305 also draws the permanent magnet 1308 which is attracted to the bottom of plunger 1305, resulting in the pair moving against spring 1306 together. This stretches the membrane 1303, which is sandwiched in between, up in a tent-like manner forming a channel between inlet channel 1307 and outlet channel 1309, which provides a fluid passageway under positive and negative pressures. In other words, in negative pressure circumstances, the permanent magnet 1308 serves to push membrane 1303 away from inlet and outlet channels 1307, 1309, so as to counteract the negative pressure.

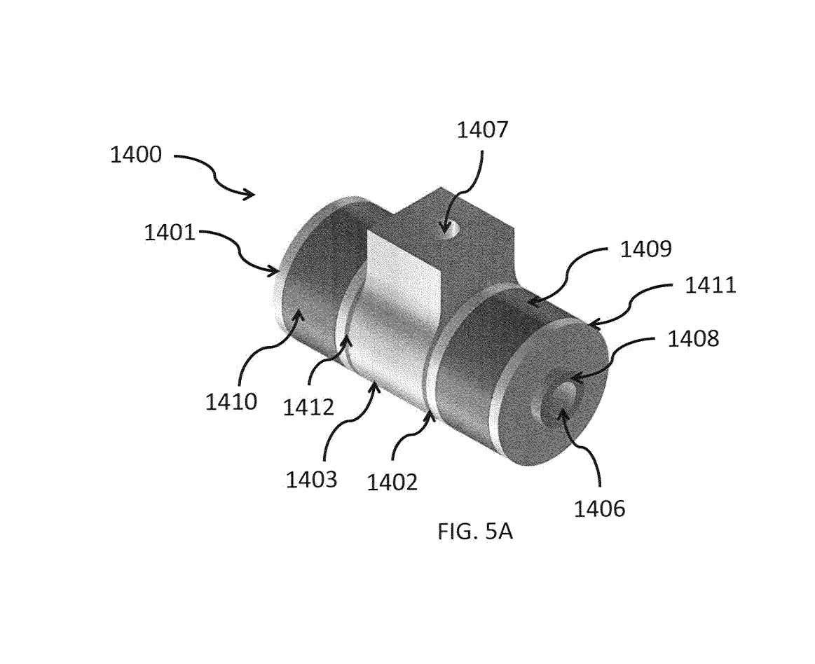

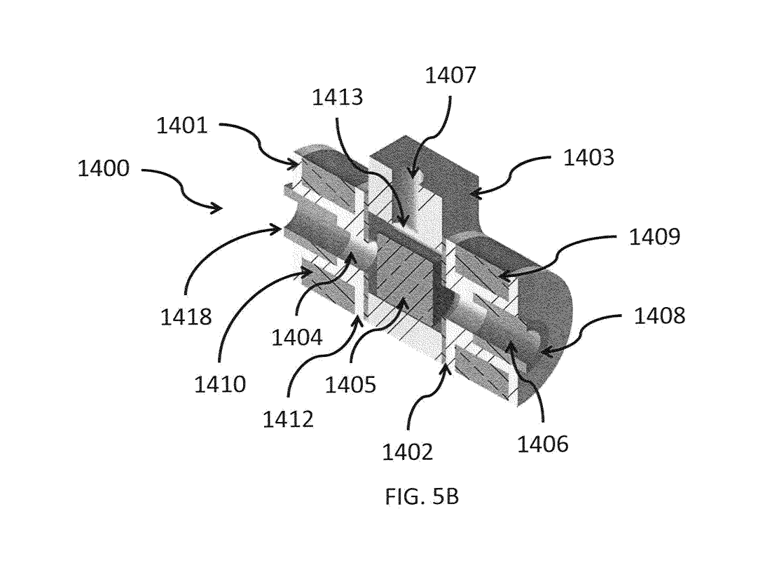

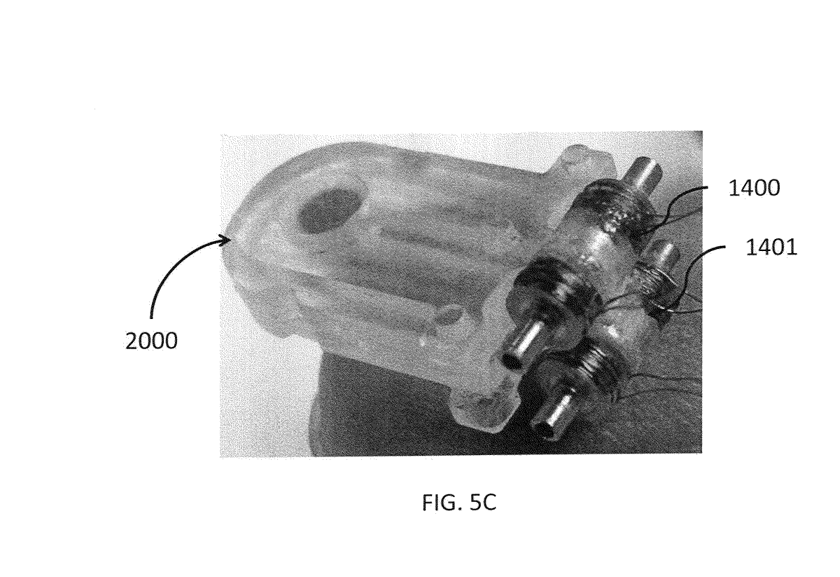

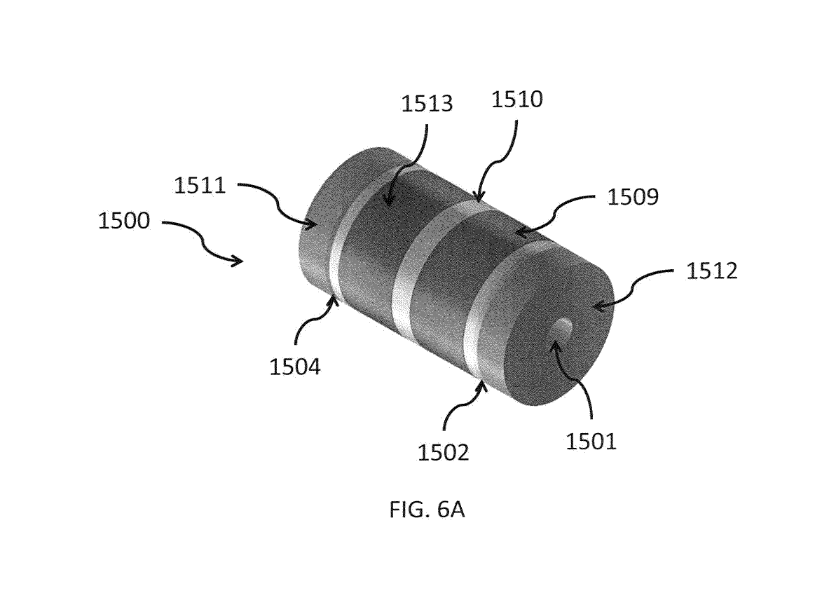

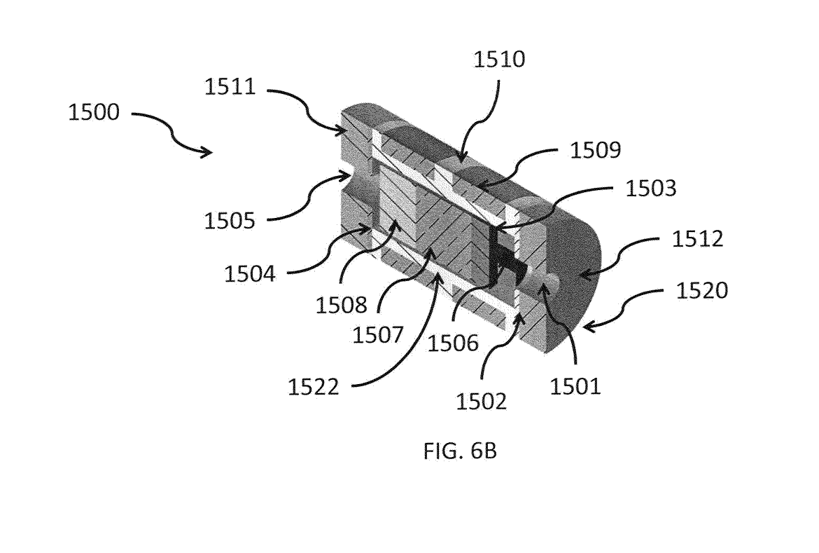

With reference now to FIGS. 5A-6B, embodiments of a bi-stable valve, according to the present disclosure is described. As shown in FIG. 5A, bi-stable valve 1400 may include a valve body 1403. The valve body 1403 may include any suitable configuration and may be made of any suitable material known in the art. In one embodiment, the valve body may include a permanent magnet 1405, two electromagnets 1409, 1410 wound on geometrically custom bobbins 1401, 1411, two silicone based seals 1402, 1412, and two ports 1408, 1418 with a relatively high magnetic permeability, as discussed above. The ports 1408, 1418 may have a substantially tube-like configuration. In addition, the ports 1408, 1418 may be made of any suitable magnetic material, including, e.g., stainless steel. The electromagnets 1409 and 1410 provide the forces necessary for actuating the valve and the ports 1408, 1418 provide the holding force required for bi-stable operation, as discussed in greater detail below. The unique geometry and configuration of the components described herein allow for the construction of an extremely compact and small three-way bi-stable micro-valve 1400. In addition, although the discussed embodiment of valve 1400 is a three-way valve, those of ordinary skill in the art will understand that the principles described herein may be utilized in connection with a suitable two-way or multi-way valve, as desired.

Under normal non-energized conditions, the magnet 1405 will be attracted more to one of the two ports 1408, 1418 and magnetically secured against the corresponding seal 1402, 1412. In this initial configuration, the ports 1408, 1418 that attracts magnet 1405 will be blocked by the magnet 1405, thereby preventing fluid flow through the respective port 1408, 1418. At the same time, a fluid flow path will be open between the other port 1408, 1418 and the center port 1407 in the body allowing fluid flow through a semicircular channel in the upper portion 1413 of valve body 1403. Although the depicted embodiment includes three ports, those of ordinary skill in the art will understand that a greater or less number of ports may be provided in valve body 1403.

When energized, each electromagnet 1409, 1410 generates an individual magnetic field and the pair of electromagnets 1409, 1410 are configured to produce opposing magnetic fields. That is, either the north poles of each coil face each other, or the south poles of each coil face each other, depending on the polarity of the energizing current. A momentary pulse of current through the electromagnets 1409, 1410 will generate a magnetic field that will cause the polarity of electromagnets 1409, 1410 to reverse. At this point, the magnet 1405 will now be more strongly attracted to the other of ports 1408, 1418, thereby causing the magnet 1405 to magnetically secure against the other of seals 1402, 1412. Thus, fluid flow through the port 1408, 1418 currently attracting magnet 1405 will be blocked. Simultaneously, a fluid flow path will open between the previously closed port 1408, 1418 and the center port 1407.

Momentarily energizing the electromagnets 1409, 1410 to once again reverse their polarities will cause magnet 1405 to be attracted to the other of ports 1408, 1418. The benefit of the described actuation method includes alleviating the need for a continuous current stream for valve actuation, which reduces the overall energy required to operate the valve, thereby making the described valve well-suited for relatively smaller battery-powered devices.

Turning to FIG. 5C, there is depicted an exemplary implementation of the bi-stable valve embodiment of FIGS. 5A-5B, FIG. 50 illustrates first and second bi-stable valves 1400 and 1401 operably coupled to a medicament cartridge 2000 having a plurality of medicament reservoirs (not shown). The medicament cartridge 2000 may include one or more of the features of the cartridge assembly described in U.S. patent application Ser. No. 13,448,013, entitled MEDICATION DELIVERY DEVICE WITH MULTI-RESERVOIR CARTRIDGE SYSTEM AND RELATED METHODS OF USE, filed Apr. 16, 2012, the entirety of which is incorporated herein by reference. In the depicted example, cartridge 2000 may include two medicament reservoirs, and each of valves 1400 and 1401 may be configured to meter medicament delivery from one of the two reservoirs.

With reference to FIGS. 6A-6B, a valve 1500 includes a valve body 1520 housing a magnetic plunger 1522. Magnetic plunger 1522 may include a permanent magnet 1507. The magnetic plunger 1507 may be disposed in a cavity formed by the inner surfaces of an electromagnetic spool 1510 having electromagnets 1509 and 1513 mounted thereon. The magnetic plunger 1522 may include three discrete parts secured together. As shown in FIG. 6B, e.g., a permanent magnet 1507 may be disposed between a hard polymer spacer 1508 and a hard polymer microchanneled crown 1506. The crown 1506 may define a fluid flow channel therethrough. Those of ordinary skill in the art will understand that the spacer 1508 and the crown 1506 may be fabricated from any suitable material. In some embodiments, the permanent magnet 1507 may be bonded by a suitable adhesive to spacer 1508 and crown 1506. In other embodiments, the permanent magnet 1507, spacer 1508, and crown 1506 may be made integrally of a one-piece construction. For example, spacer 1508 and crown 1506 may be molded together with permanent magnet 1507 disposed therebetween.

Under normal non-energized conditions, the magnet 1507 will be attracted more strongly to one of two ports 1501 and 1505 and magnetically secured against the corresponding seal 1504 or plastic spacer 1502. Ports 1501 and 1505 may be defined in structures 1511, 1512 secured to electromagnetic spool 1510. Effectively, therefore, magnet 1507 may be attracted to one of structures 1511, 1512. As described above, ports 1501 and 1505 may be made of any suitable magnetic material. In some embodiments, ports 1501 and 1505 may be defined by, e.g., stainless steel tubes disposed in structures 1511, 1512. In other embodiments, structures 1511, 1512 themselves may be made of a magnetic material. For example, structures 1511, 1512 may be stainless steel plates. For purposes of discussion, the initial position of magnet 1507 is described such that the hard polymer spacer 1508 of the magnetic plunger 1522 is magnetically held against the seal 1504. The corresponding port 1505, and its channel, will therefore be blocked and fluid flow through this portion of the valve 1500 will be prevented.

When the electromagnets 1509 and 1513 are energized, the coil of each electromagnet 1509, 1513 generates an individual magnetic field and the two electromagnets 1509, 1513 are configured to produce opposing magnetic fields. That is, either the north poles of each electromagnet 1509, 1513 face each other, or the south poles of each electromagnet 1509, 1513 face each other, depending on the polarity of the energizing current. A momentary pulse of current through the oils of electromagnets 1509, 1513 will generate a magnetic field that will cause the permanent magnet 1507 to reverse polarity and become attracted to the other port 1501. In this second configuration, the hard polymer microchanneled crown 1506 will be in contact with the plastic spacer 1502. That is, the magnet 1507 will now be more attracted to the other structure 1512 if it was first attached. The microchanneled crown 1506 is configured to mate with the plastic spacer 1502 in such a way that a pathway for fluid flow through the valve body 1520 is established. In other words, in this configuration, the bi-stable valve 1500 allows fluid flow between the two ports 1501 and 1505. That is, fluid may flow into the valve at, e.g., port 1501, through the channel(s) of crown 1506, in between an inner wall of valve body 1520 and the outer walls of plunger 1522 until the fluid reaches port 1505, and vice versa.

Momentarily energizing the electromagnets 1509 and 1513 in the reversed polarity will cause magnet 1507 to become attracted to the other of ports 1501 and 1505, thereby forming a bi-stable electromagnetically controlled valve 1500. The benefit of this actuation method also includes alleviating the need for providing a continuous current stream for actuation. This dramatically reduces the overall energy required to drive the valve and thereby makes this valve well suited for small battery powered devices.

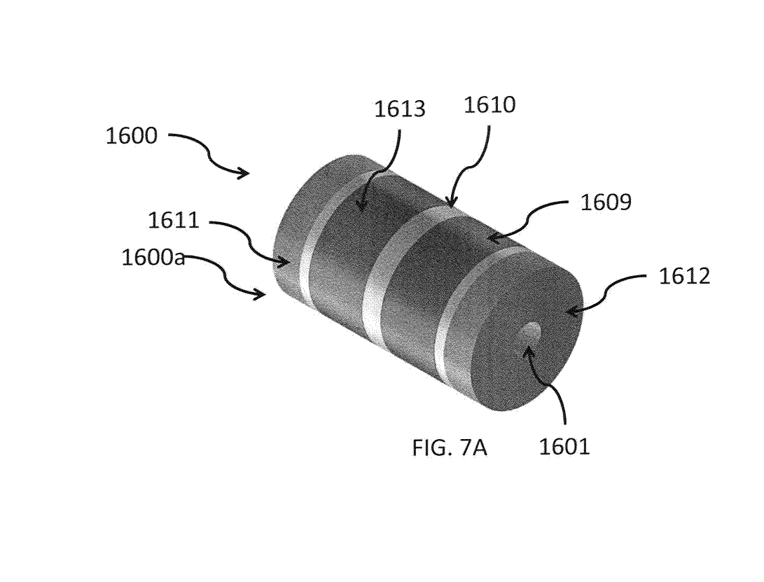

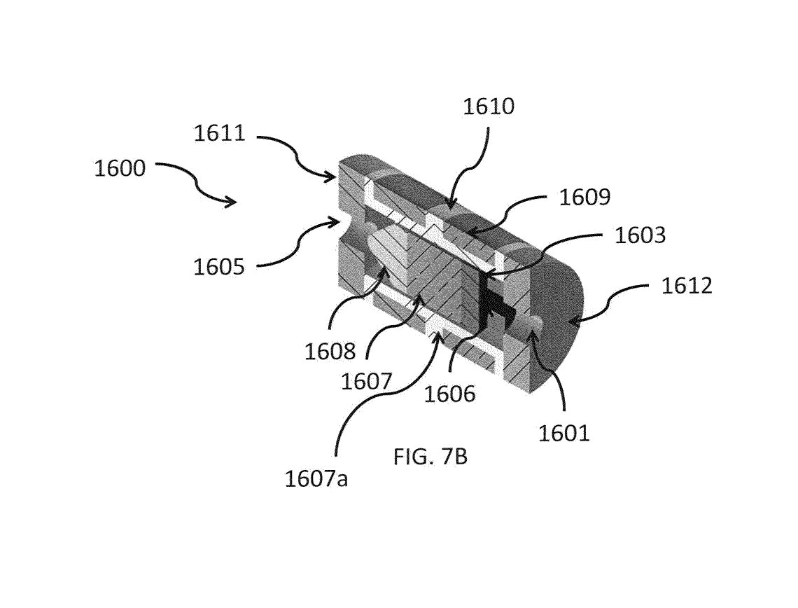

Referring to FIGS. 7A-7B, there is depicted a further embodiment of a valve 1600, in accordance with the principles of the present disclosure. As alluded to above valve 1600 may have one or more of the features described in connection with the other embodiments discussed herein. Valve 1600 may include a valve body 1600a. Valve body 1600a may include any suitable configuration. For example, valve body 1600a may be substantially cylindrical, as depicted in, e.g., FIG. 7A. In one embodiment, valve body 1600a may include an electromagnetic spool 1610. One or more electromagnets 1609 and 1613 may be received on electromagnetic spool 1610. Valve body 1600a may further define a cavity therein. The cavity may be formed by the inner surfaces of electromagnetic spool 1610. A plunger 1607a may be disposed within the cavity of the valve body 1600a.

The plunger 1607a may include three parts which are secured together by, e.g., an adhesive. In one embodiment, the plunger 1607a may include a permanent magnet 1607, a seal 1608, and a microchanneled crown 1606. Under non-energized conditions, the magnet 1607 will be attracted more strongly to one of the two ports 1611 and 1612 and magnetically secured against the corresponding port. Ports 1611 and 1612 may be made of any suitable magnetic material. For example, in one embodiment, ports 1611 and 1612 may be made of stainless steel. Alternatively, ports 1611 and 1612 may be made of a suitable non-magnetic material. However, in order to attract magnet 1607, ports 1611 may include a portion made of a suitable magnetic material.

Seal 1608 may include any suitable configuration. For example, in one embodiment, seal 1608 may include a substantially conical configuration. In other embodiments, however, seal 1608 may include a substantially cylindrical configuration. Still further, seal 1608 may include a substantial spherical configuration. Furthermore, seal 1608 may be made of any suitable material. For example, seal 1608 may be made of a suitable elastomeric material, which may be relatively softer than the materials used to fabricate port 1611. The elastomeric material may be capable of deforming to improve sealing function. Furthermore, microchanneled crown 1606 may include a number of channels disposed therein to allow fluid flow therethrough. In addition, microchanneled crown 1606 may be fabricated of suitable polymeric material, which may be relatively harder than the elastomer used to fabricate seal 1608.

For purposes of discussion, the plunger 1607a may be in an initial position such that seal 1608 is magnetically attracted to port 1611. Consequently, channel 1605 of port 1611 will be blocked preventing fluid flow through the valve.

When one or more of electromagnets 1609 and 1613 are energized, each of the electromagnets 1609 and 1613 may generate an individual magnetic field. In one embodiment, the generated magnetic fields may be opposite to one another. For example, either the north poles of each electromagnet may face each other, or the south poles of each electromagnet may face each other, depending on the polarity of the energizing current. That is, each electromagnet 1609 and 1613 may generate a field of similar polarity.

A momentary pulse of current through the electromagnets 1609 and 1613 will generate a magnetic field which will cause the magnet 1607 to reverse polarity and flip to the other side within the valve body 1600a. At this point, the magnet 1607 will now be more strongly attracted to the other port 1612, ensuring that it is now magnetically secured thereto. In this second configuration, the channels of microchanneled crown 1606 will be in fluid communication with port 1612. That is, the microchanneled crown 1606 mates with the port 1612 in such a way that a pathway for fluid flow is provided from channel 1605 in port 1611, through valve body 1600a, and to channel 1601 in port 1612. The fluid may flow within the valve body 1600a around plunger 1607. In this configuration, the valve 1600 may allow fluid flow between the two ports 1611 and 1612. Those of ordinary skill will understand that fluid may flow from channel 1601 to channel 1605 as well.

Momentarily energizing the magnets 1609 and 1613 again in the reversed polarity will flip the magnet back to the other side (i.e., the side of port 1611), thereby forming an electromagnetically controlled bi-stable latching proportioning valve 1600. The benefit of this actuation method is alleviating the need for a continuous current to provide actuation. This dramatically reduces the overall energy required to drive the valve and thereby makes this valve well suited for small battery-powered devices.

Furthermore, by incorporating a positional feedback sensor and a controller (not shown), the frustum or conical design of the seal 1608 may enable proportioning valve operation. For example, in the fully closed condition, the seal 1608 may be in contact with port 1611. That is, in embodiments where the seal 1608 is configured as cone, a substantial portion of seal 1608 may be received in channel 1605, thereby fully blocking fluid flow therethrough. As the seal 1608 is slowly electromagnetically pulled away from port 1611, initially a small cross-sectional area of channel 1605 becomes available for fluid flow. As the gap between the seal 1608 and port 1611 is increased, the cross-sectional area of channel 1605 available for fluid flow also increases. Conversely, the elastomer cone seal 1608 can also be driven back towards port 1611 reducing the cross-sectional area available for fluid flow. The overall system therefore allows for full proportional control of the fluid flow while also offering bi-stable latching operation. Furthermore, as those of ordinary skill in the art will recognize, the valve 1600 may be provided with a mechanism for selectively controlling the position of seal 1608 relative to channel 1605. For example, valve body 1600a may include a mechanical stop that may be actuated to maintain a portion of seal 1608 within channel 1605. Valve 1600 may also be provided with a suitable mechanism for controlling the speed of travel of plunger 1607 within valve body 1600.

While principles of the present disclosure are described herein with reference to illustrative embodiments for particular applications, it should be understood that the disclosure is not limited thereto. Those having ordinary skill in the art and access to the teachings provided herein will recognize additional modifications, applications, embodiments, and substitution of equivalents all fall within the scope of the embodiments described herein. Accordingly, the invention is not to be considered as limited by the foregoing description.

We claim:

1. A valve assembly, comprising:
a valve body defining a housing for receiving a plunger, wherein the valve body includes an inlet channel and an outlet channel in selective fluid communication with the inlet channel;
a deflectable membrane operably coupled to the plunger, wherein the membrane is configured to selectively prevent fluid flow from the inlet channel to the outlet channel; and
an electromagnet for controlling a position of the plunger.

2. The valve assembly of claim 1, wherein the valve body includes a first housing member and a second housing member.

3. The valve assembly of claim 2, wherein the inlet and outlet channels are defined in the second housing member.

4. The valve assembly of claim 1, wherein the inlet channel includes a plurality of inlet channels, and the outlet channel includes a plurality of outlet channels.

5. The valve assembly of claim 2, wherein the deflectable membrane is disposed between the first and second housing members.

6. The valve assembly of claim 2, further including a resilient member configured to bias the plunger towards the deflectable membrane.

7. The valve assembly of claim 6, wherein the resilient member is disposed between the first housing member and the electromagnet.

8. The valve assembly of claim 1, wherein the plunger includes a plate operably coupled to a curved actuating portion.

9. The valve assembly of claim 2, further comprising a return spring configured to bias the deflectable membrane towards the plunger.

10. The valve assembly of claim 9, wherein the return spring is disposed in the second housing member.

11. The valve assembly of claim 1, further comprising a permanent magnet, wherein the membrane is disposed between the permanent magnet and the plunger.

12. A valve assembly, comprising:
a valve body defining an inlet port and a plurality of outlet ports;
a plurality of electromagnets, wherein each of the plurality of outlet ports extends through one of the plurality of electromagnets; and
a permanent magnet movably disposed within the valve body, wherein the permanent magnet is configured to divert fluid flow from the inlet port to one of the plurality of outlet ports.

13. The valve assembly of claim 12, wherein the permanent magnet is configured to be attracted to one of the plurality of outlet ports.

14. The valve assembly of claim 13, wherein, upon application of an electrical pulse, the valve assembly is configured to reverse a polarity, causing the permanent magnet to become attracted to the another of the plurality of outlet ports.

15. The valve assembly of claim 12, wherein the permanent magnet is configured to selectively prevent fluid flow through one of the plurality of outlet ports.

16. A valve assembly, comprising;
a valve body having a first end and a second end, wherein the first end defines an inlet port and the second end defines an outlet port;
a magnetic plunger assembly disposed in a cavity defined by the valve body and configured to move between a first position and a second position, wherein, in the first position, the magnetic plunger assembly is configured to prevent fluid flow into the valve body from the inlet port; and
an electromagnet disposed about the cavity, wherein selective actuation of the electromagnet controls a position of the magnetic plunger assembly.

17. The valve assembly of claim 16, wherein, in the second position, the magnetic plunger is configured to allow fluid to flow from the inlet port to the outlet port.

18. The valve assembly of claim 16, wherein, in the first configuration, the magnetic plunger is attracted to the first end of the valve body.

19. The valve assembly of claim 18, wherein energizing the electromagnet causes the magnetic plunger assembly to become attracted to the second end of the valve body.

20. The valve assembly of claim 16, wherein the electromagnet includes a plurality of electromagnets.

21. The valve assembly of claim 16, wherein energizing the electromagnet reverses a polarity of the magnetic plunger assembly.

22. The valve assembly of claim 16, wherein the magnetic plunger assembly includes a permanent magnet having a first end and a second end, a seal disposed at the first end, and a crown disposed at the second end.

23. The valve assembly of claim 22, wherein the seal and the crown are adhesively secured to the permanent magnet.

24. The valve assembly of claim 22, wherein the seal includes a substantially conical shape.

25. The valve assembly of claim 24, wherein the seal comprises a deformable material.

26. The valve assembly of claim 22, wherein the crown includes at least one channel configured for fluid flow therethrough.

27. The valve assembly of claim 22, wherein the permanent magnet, the seal, and the crown are configured to move together within the valve body.

28. The valve assembly of claim 16, wherein a portion of the valve body comprises a material configured to sustain a magnetic field.

29. The valve assembly of claim 16, wherein a portion of the valve body comprises a material having a high magnetic permeability.

30. A medicament delivery apparatus, comprising:
a first reservoir including a first medicament;
a second reservoir including a second medicament different from the first medicament;
a first valve configured to meter delivery of the first medicament from the first reservoir; and
a second valve configured to meter delivery of the second medicament from the second reservoir, wherein one of the first and second valves comprises:
a valve body defining a housing for receiving a plunger, wherein the valve body includes an inlet channel and an outlet channel in selective fluid communication with the inlet channel;
a deflectable membrane operably coupled to the plunger, wherein the membrane is configured to selectively prevent fluid flow from the inlet channel to the outlet channel; and
an electromagnet for controlling a position of the plunger.

31. A medicament delivery apparatus, comprising:
a first reservoir including a first medicament;
a second reservoir including a second medicament different from the first medicament;
a first valve configured to meter delivery of the first medicament from the first reservoir; and
a second valve configured to meter delivery of the second medicament from the second reservoir, wherein one of the first and second valves comprises:
a valve body having a first end and a second end, wherein the first end defines an inlet port and the second end defines an outlet port;
a magnetic plunger assembly disposed in a cavity defined by the valve body and configured to move between a first position and a second position, wherein, in the first position, the magnetic plunger assembly is configured to prevent fluid flow into the valve body from the inlet port; and
an electromagnet disposed about the cavity, wherein selective actuation of the electromagnet controls a position of the magnetic plunger assembly.

32. The medicament delivery apparatus of claim 31, wherein, when the magnetic plunger assembly is in the second position, fluid is permitted to flow from the inlet port, through the valve body, and out of the outlet port.
